(19) United States
(12) Plant Patent Application Publication
(10) Pub. No.: US 2006/0090230 P1
(43) Pub. Date: Apr. 27, 2006

(54) Malus 'Summer Wonder'

(50) MALUS PUMILA; APPLE

(76) Inventor: Kenneth Ray Gill, Baileyton, AL (US)

Correspondence Address: KENNETH GILL;GILL NURSERY, INC., 100 COUNTY ROAD 1595, BAILEYTON, AL, 35019, US

(21) Appl. No.: 10/972,303

(22) Filed: Oct. 25, 2004

(51) Int. Cl. A01H 5/00

(52) U.S. Cl. PLT/173

(57) ABSTRACT

Mr. Kenneth Gill of Gill Nursery in Baileyton, Ala. introduces a cultivated seedling selection of Malus ‘Antoanovka” named ‘Summer Wonder’ which offers the landscape industry a unique disease resistant purple-leafed crabapple with stable color in southern climates (zones 7b/8a) with pinkishreddish purple flowers and desirably small fruit for the urban landscape.

### BACKGROUND OF INVENTION

A new and distinct genetic variation from an open pollinated seedling of the crabapple, Malus baccata×Malus pumila ‘Antonovka”, was discovered in the nursery production seedling row at Gill Nursery by the sole inventor, Mr. Kenneth Gill in Baileyton, Alab. on Jun. 5, 1998. This cultivar is of Russian origin and is used in the nursery industry as a hardy root stock for Malus cultivars. Seeds from this open pollinated cultivar were sown and cultivated in the field for use as budding root stock. The ‘Antonovka’ seed came from Central Washington Nursery, 11641 Rd 10 NW, Quincy, Wash., 98848 collected from green leaf forms of ‘Antonovka’. The new Malus cultivar named ‘Summer Wonder’ by the discoverer, Mr. Gill, has been observed and evaluated for six years as a landscape plant and has maintained its unique purple leaf characteristics. It has not been sold, shared or released and has not been made public in any publications. Summer Wonder crabapple has been successfully asexually propagated by budding in September and October on Malus domestica and ‘Antonovka’ open pollinated seedling rootstock in Cullman, Ala. at the inventor's nursery. Budding success rate has been consistent at about 80-90% success rate. The clonal progeny of the parent plant are identical to the parent and offer the same distinct characteristics. The clonal progeny of the parent plant are true to type, identical to the parent and offer the same distinct characteristics.

### SUMMARY OF INVENTION

unique characteristics expressed by this new cultivar are stable deep purple leaves which are unique to this normally green cultivar. ‘Summer Wonder’ has landscape desirable, very small purple fruit with reddish/purple flowers. Bottom of the leaves are also a lighter purple. The pith of the branches are also purple in color. There are other purple leaf crabapple cultivars from different Malus species but few, if any, that maintain the continuous purple color in the heat of climatic zones 7b/8a in Cullman, Ala. Malus ‘Robinson” and M. ‘Profusion’ are cultivars that offer red leaves at emergence but fade to green during the summer. (See attached photo) M. ‘Royalty’ and M. ‘Velvet Pillar’ are two other cultivars noted for their purple leaves. ‘Royalty’ (pumila×baccata) is an open pollinated seedling of Rosybloom and has red fruit and is highly susceptible to fireblight and scab. ‘Velvet Pillar’, a patented cultivar (U.S. Plant Pat. No. 4785) by Simpson also is noted for its purple leaves, single pink flowers and reddish fruit, listed as fair to good disease resistance. It is an upright selection 20 feet (6 m) high×14 (4.3 m) wide. ‘Summer Wonder’ crabapple selection has also exhibited unique resistance to typical crabapple diseases of cedar-apple rust (Gymnosporangium juniperi-virginianae), powdery mildew (Podosphaera leucotricha), apple scab (Venturia inaequalis) and fire blight (Erwinia amylovora) where clones of the tree were exposed to innoculi of the pathogens in surrounding fields of other species and cultivars of Malus. These diseases were evident in surrounding fields where other crabapples were moderately or severely affected. These unique and distinct characteristics separate ‘Summer Wonder’ from all other plants within its botanical and market class.

### DESCRIPTION OF PHOTOS

Photos represent flower and fruit color, 3 year old plant in the field, fall and spring foliage and foliage of M. ‘Summer Wonder” compared to M. ‘Robinson’ and M. ‘Profusion’ under summer heat conditions.

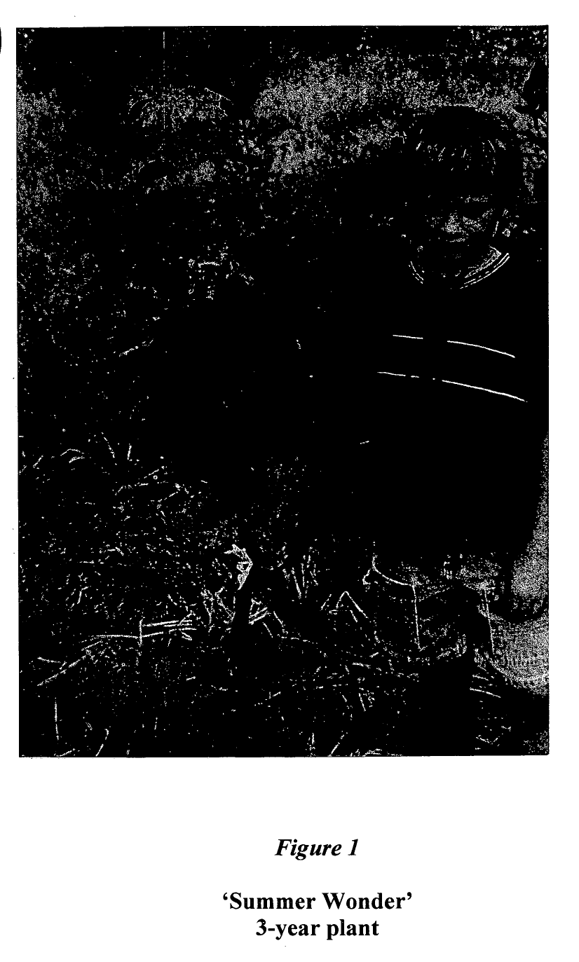

FIG. 1, ‘Summer Wonder’ 3 year plant;

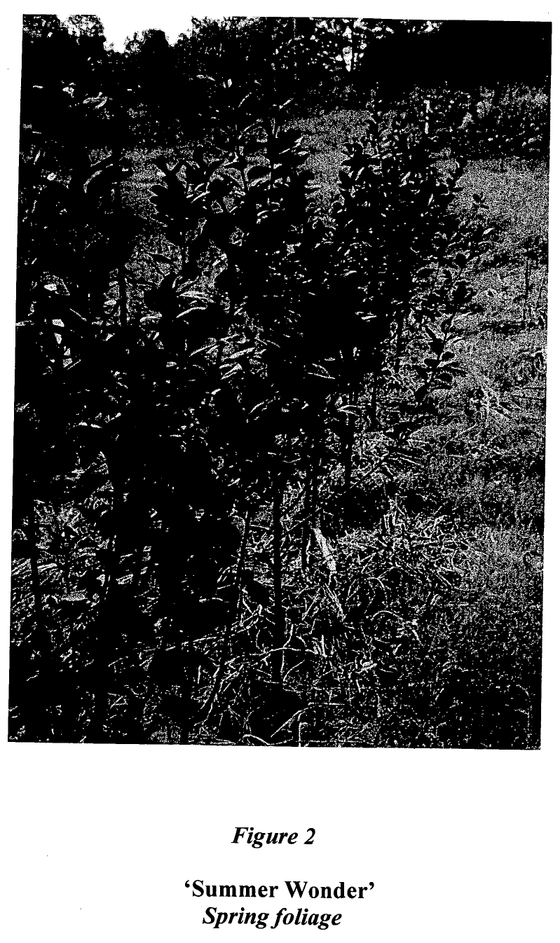

FIG. 2, ‘Summer Wonder’ Spring foliage;

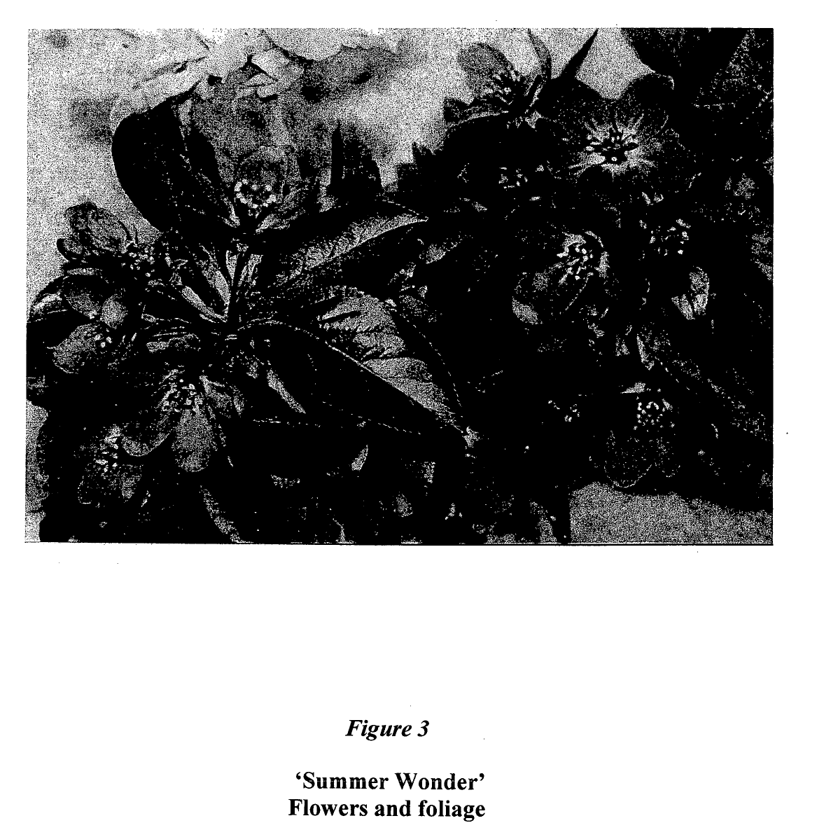

FIG. 3, ‘Summer Wonder’ Flowers and foliage;

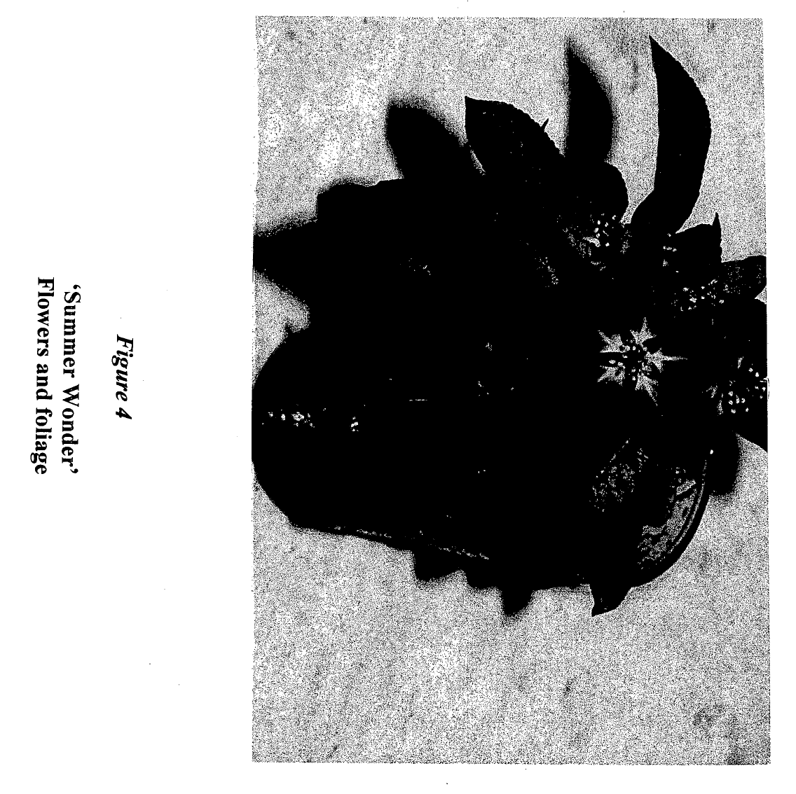

FIG. 4, ‘Summer Wonder’ Flowers and foliage;

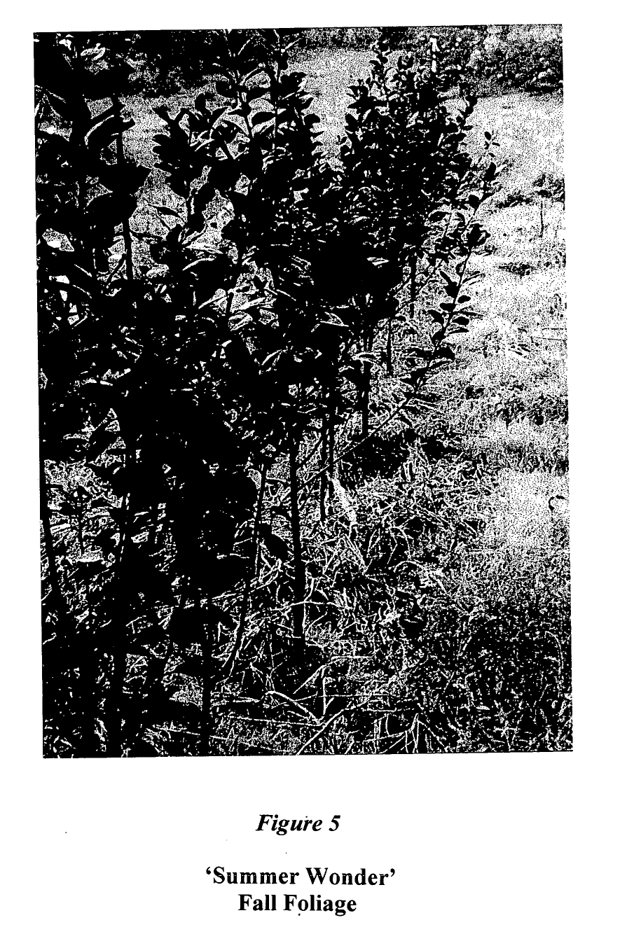

FIG. 5, ‘Summer Wonder’ Fall Foliage;

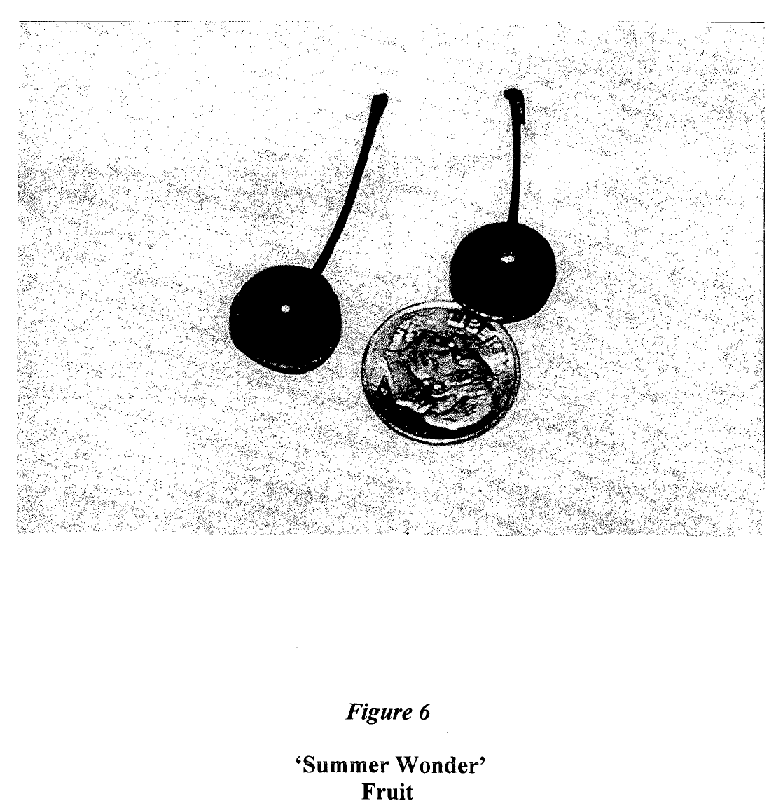

FIG. 6, ‘Summer Wonder’ Fruit;

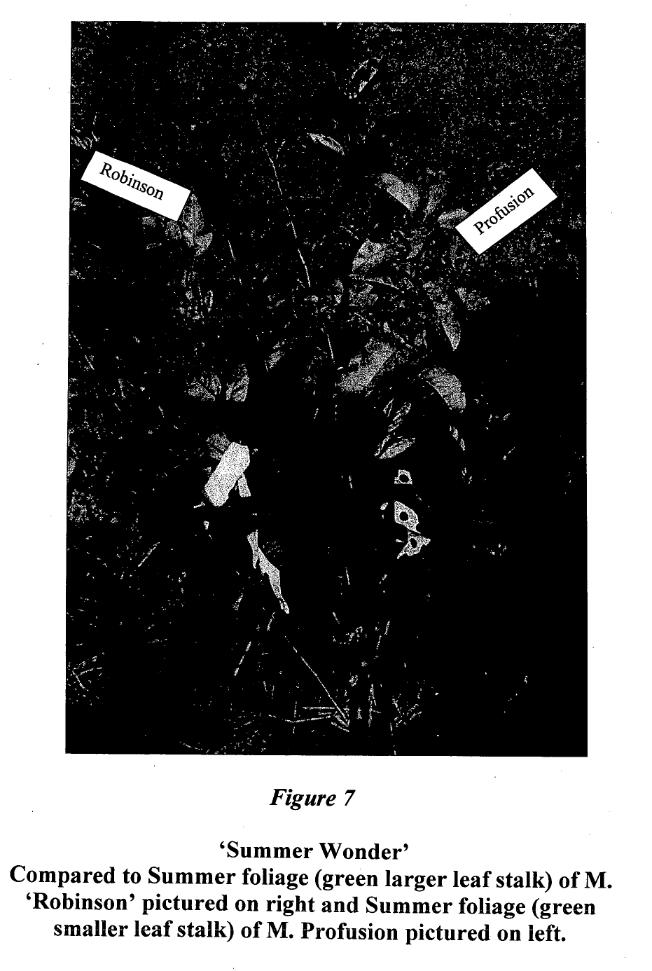

FIG. 7, ‘Summer Wonder’ Comparison to summer foliage of other crabapple varities;

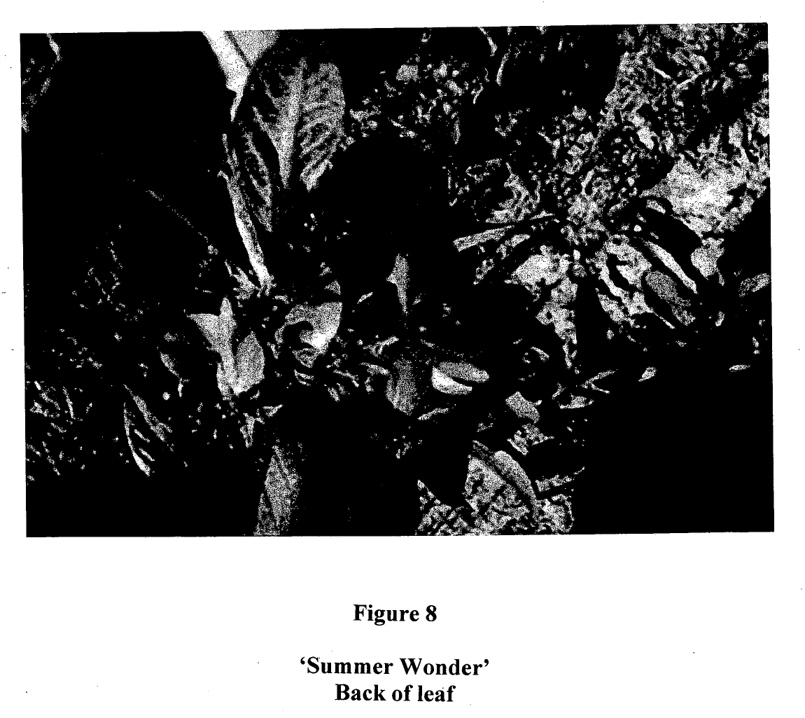

FIG. 8, ‘Summer Wonder’ back of leaf.

### BOTANICAL DESCRIPTION OF THE PLANT

The original specimen of this 6 year old new Malus cultivar is now 8 feet (2.4 meters) tall and 5 feet ( 1.5 meters) wide with a trunk diameter of 3 inches (7.6 cm). Average growth is 2 feet (0.6 meters) per year. Mature leaf measurements range from 2.6 inches (6.6 cm) to 3.2 inches (7.6 cm) long to 1.5 inches (3.8 cm) to 1.7 (4.3 cm) wide. The mean leaf size is 2.9 inches (7.4 cm) long to 1.6 inches (4.0 cm) wide. The leaf is a typical shape of the species. Leaves are alternate, ovate, with acute bases and apices and serrate margins. Stems are dark purple with prominent lenticels which average 15 to 22 per stem section 2.5 cm and 0.25 to 1.0 mm in diameter. The unique color of the leaves as measured by the Minolta Spectrophotometer (Model CM 2002)

| Name | L* | a* | b* | C* | h |
|---|---|---|---|---|---|
| Leaf Top | 27.8911 | 5.9290 | 3.2092 | 6.7418 | 28.4259 |
| Leaf Back | 33.1136 | 10.5724 | 4.7685 | 11.5980 | 24.2770 |
| Flower | 34.6821 | 26.2700 | 1.2371 | 26.2991 | 2.6962 |
| Stem Wood | 56.6625 | 3.7518 | 2.7246 | 4.6367 | 35.9872 |

Single flowers occur in March and bloom for 2 weeks. Buds are reddish purple in color and open to the characteristics pink/purple color designated by color ratings above. Individual flowers have 5 petals and average ½ inch (1.3 cm)×¼ inch (0.6 cm) wide and are arranged in clusters of 4 to 6 flowers per cluster. This contrasts with the typical small white flowers of the seedling ‘Antonovka.’ There is a strong pleasant fragrance. Fruit diameter averages 0.6 inches (1.6 cm). Fruit color is deep purple and listed in the table above. Bark has a typical color of the M. ‘Antonovka’ parent. Mature tree exhibits a spreading with branches extending from the trunk at typical angles ranging from 75 degrees to 35 degrees.

1. Malus ‘Summer Wonder’ is an easily propagated, unique landscape cultivar introduction of Malus×“Antonovkaantanovka with heat tolerant, seasonally stable purple leaves with pinkishreddish purple flowers and small purple fruit with disease resistant qualities for climatic zones 7b/8a.
